(19) United States
(12) Patent Application Publication
(10) Pub. No.: US 2022/0226341 A1
(43) Pub. Date: Jul. 21, 2022

(54) Pharmaceutical Composition for Alzheimer's Disease

(71) Applicant: Kyoto University, Kyoto-shi, Kyoto (JP)

(72) Inventors: Masatoshi Hagiwara, Kyoto (JP); Masahiko Ajiro, Kyoto (JP)

(73) Assignee: Kyoto University, Kyoto-shi, Kyoto (JP)

(21) Appl. No.: 17/613,303

(22) PCT Filed: May 22, 2020
(86) PCT No.: PCT/JP2020/020280

§ 371 Date: Nov. 22, 2021

(30) Foreign Application Priority Data
May 23, 2019 (JP) 2019-096908

(51) Int. Cl. A61K 31/55; A61K 31/4545; A61K 31/505; A61K 9/00; A61P 31/12

(52) U.S. Cl. CPC A61K 31/55; A61K 31/4545; A61P 31/12; A61K 9/0019; A61K 31/505

(57) ABSTRACT

A novel pharmaceutical composition and method that enable the treatment, amelioration, prevention, and/or suppression of progression of Alzheimer's disease are provided.

Provided is a pharmaceutical composition for preventing, treating, suppressing the progression of, and/or ameliorating Alzheimer's disease, the pharmaceutical composition containing, as an active ingredient, a compound that is capable of suppressing the activation or growth of virus in the brain as one of factors that cause the onset or progression of Alzheimer's disease. Also provided is a method for preventing, treating, suppressing the progression of, and/or ameliorating Alzheimer's disease, the method including administering an effective amount of the compound to a subject.

### TECHNICAL FIELD

The present disclosure relates to a pharmaceutical composition for treatment, amelioration, prevention, and/or suppression of progression of Alzheimer's disease.

### BACKGROUND ART

It is said that Alzheimer's disease accounts for 60 percent of dementia cases. As a therapeutic drug for Alzheimer's disease, however, there are currently only symptom improving drugs intended to alleviate clinical symptoms of Alzheimer's disease. Therefore, the development of an effective pharmacotherapy has been desired.

The cause of Alzheimer's disease has not been completely explained yet, and patients have various conditions. Therefore, various targets of drug development have been studied. For example, based on the overexpression of the gene of dual-specificity tyrosine phosphorylation-regulated kinase 1A (DYRK1A), which is a kind of tau phosphorylation enzymes, in the brains of Alzheimer's disease patients, studies have been made on a therapeutic drug for Alzheimer's disease with use of a compound that inhibits phosphorylation enzyme activity of DYRK1A (Non-patent Document 1).

### PRIOR ART DOCUMENT

### Non-Patent Document

[Non-Patent Document 1] Ogawa et al., Nature Communications 1, 86 (2010) 1-9.

### SUMMARY OF THE INVENTION

### Problem to be Solved by the Invention

The present disclosure, in one aspect, provides a novel pharmaceutical composition and method that enable the treatment, amelioration, prevention, and/or suppression of progression of Alzheimer's disease.

### Means to Solve the Problem

The present disclosure, in one aspect, relates to a pharmaceutical composition for preventing, treating, suppressing the progression of, and/or ameliorating Alzheimer's disease, the pharmaceutical composition containing, as an active ingredient, a compound that is capable of suppressing the activation or growth of virus in the brain as one of factors that cause the onset or progression of Alzheimer's disease.

The present disclosure, in another aspect, relates to a method for preventing, treating, suppressing the progression of, and/or ameliorating Alzheimer's disease, the method including administering, to a subject, an effective amount of a compound that is capable of suppressing the activation or growth of virus in the brain as one of factors that cause the onset or progression of Alzheimer's disease.

### Effect of the Invention

With the present disclosure, in one aspect, it is possible to provide a novel pharmaceutical composition and method that enable the treatment, amelioration, prevention, and/or suppression of progression of Alzheimer's disease.

### BRIEF DESCRIPTION OF DRAWINGS

FIG. 1 is a graph showing a comparison of fold-change against the amounts of human herpesvirus type 6A (HHV-6A) in antiviral test against HHV-6A.

FIG. 2 is a graph showing a comparison of fold-change against the amounts of human herpesvirus type 7 (HHV-7) in antiviral test against HHV-7.

### MODE FOR CARRYING OUT THE INVENTION

[Pharmaceutical Composition for Prevention, Treatment, Suppression of Progression, and/or Amelioration of Alzheimer's Disease]

The present disclosure, in one aspect, relates to a pharmaceutical composition for preventing, treating, suppressing the progression of, and/or ameliorating Alzheimer's disease (the pharmaceutical composition of present disclosure), the pharmaceutical composition containing, as an active ingredient, a compound that is capable of suppressing the activation or growth of virus in the brain as one of factors that cause the onset or progression of Alzheimer's disease.

The present disclosure is based on the finding that a compound that inhibits the activity of cyclin-dependent kinase 9 (CDK9) has brain permeability (intracerebral transferability), and the compound can suppress the activation of virus in the brain. With the pharmaceutical composition of the present disclosure, in one or a plurality of embodiments, it is possible to suppress the activation of virus such as herpesvirus present in the brain. With the pharmaceutical composition of the present disclosure, which can suppress the activation of virus such as herpesvirus present in the brain, in one or a plurality of embodiments, it is possible to suppress the accumulation of an amyloid β (Aβ) aggregate in the brain, and/or the neuronal cell death caused by the accumulation of Aβ. Therefore, the pharmaceutical composition of the present disclosure, in one or a plurality of embodiments, enables the prevention, treatment, suppression of progression, and/or amelioration of Alzheimer's disease.

The compound capable of suppressing the activation or growth of virus in the brain as one of factors that cause the onset or progression of Alzheimer's disease (the compound of the present disclosure), in one or a plurality of embodiments, is a compound capable of inhibiting growth of DNA virus and/or retrovirus by inhibiting CDK9. In one or a plurality of embodiments, the DNA virus is HSV-1, HSV-2, adenovirus, or human papillomavirus (HHV-6A, HHV-7, etc.). In one or a plurality of embodiments, the compound of the present disclosure is a compound that can pass through the blood-brain barrier.

In one or a plurality of embodiments, the compound of the present disclosure is a compound expressed by Formula (I) below, or a pharmaceutically acceptable salt of the same. In one or a plurality of embodiments, as the compound expressed by Formula (I) is capable of selectively inhibit the activity of CDK9, it is capable of suppressing the replication of virus DNA, thereby suppressing the growth of the DNA virus. In one or a plurality of embodiments, the compound expressed by Formula (I), therefore, can pass through the blood-brain barrier (be transported across the blood-brain barrier), thereby reaching the cerebrum, the cerebellum, and the like. The compound expressed by Formula (I), therefore, in one or a plurality of embodiments, is capable of suppressing the activation or growth of virus, the virus being one of factors that cause the onset or progression of Alzheimer's disease, in the brain.

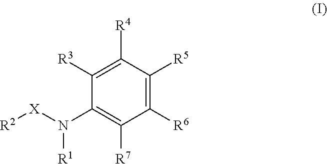

In Formula (I),

R¹ represents a hydrogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted 5 to 8 membered aryl group,

X represents —C(═O)—; —C(═S)—; —SO₂—; —C(═S)NHC(═O)—; —C(═O)NHC(═S)—; or

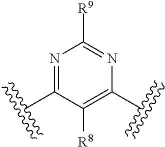

where R⁸ and R⁹ independently represent a hydrogen atom; a substituted or unsubstituted C₁₋₆ alkyl group; a C₂₋₆ alkenyl group; a C₂₋₆ alkynyl group; a halogen atom; —CN; —NH₂; or —NO₂, and a binding hand to which wavy lines are attached represents a binding part binding to the compound expressed by Formula (I),

R² represents a substituted or unsubstituted C₁₋₆ alkyl group; a substituted or unsubstituted C₂₋₆ alkenyl group; a substituted or unsubstituted C₂₋₆ alkynyl group; a substituted or unsubstituted 5 to 8 membered aryl group; a substituted or unsubstituted nitrogen-containing heterocycle; or substituted or unsubstituted fused heteroaryl group,

R³ represents a hydrogen atom; a substituted or unsubstituted C₁₋₆ alkyl group; a C₂₋₆ alkenyl group; a C₂₋₆ alkynyl group; a halogen atom; —CN; —NH₂; or —NO₂,

R⁴ represents a hydrogen atom; a halogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted C₆₋₁₀ aryl group,

R⁵ represents a hydrogen atom; a halogen atom; an amino group; or an azide group,

R⁶ represents a hydrogen atom; —CSO₂NR¹⁰R¹¹; or —CSO₂R¹²,
where
   R¹⁰, R¹¹, and R¹² independently represent a hydrogen atom, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₄ alkoxy C₁₋₄ alkyl group, a substituted or unsubstituted cycloalkyl group, a substituted or unsubstituted heterocycloalkyl group, a substituted or unsubstituted aromatic ring, or a substituted or unsubstituted heteroaryl group,
   R¹⁰ and R¹¹ each form a heterocycloalkyl group in combination with a nitrogen atom to which the same is bonded, or
   R¹⁰ and R¹¹ each form a heterocycloalkyl group in combination with a nitrogen atom to which the same is bonded, and a sulfur atom to which the nitrogen atom is bonded, and

R⁷ represents a hydrogen atom; a halogen atom; a diethylamino group; a substituted or unsubstituted nitrogen-containing heterocycloalkyl group; or a substituted or unsubstituted nitrogen-containing heteroaryl group.

The “C₁₋₆ alkyl group” in the present disclosure means a linear or branched chain alkyl group having one to six carbon atoms, which is a monovalent group derived from an aliphatic hydrocarbon having one to six carbon atoms by removing one arbitrary hydrogen atom therefrom. In one or a plurality of embodiments, the C₁₋₆ alkyl group is a methyl group, an ethyl group, a 1-propyl group, a 2-propyl group, a 2-methyl-1-propyl group, a 2-methyl-2-propyl group, an n-butyl group, an isobutyl group, a tert-butyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, a 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-butyl group, or a 2,3-dimethyl-2-butyl group.

The “C₂₋₆ alkenyl group” in the present disclosure means a linear or branched chain alkenyl group having two to six carbon atoms. In one or a plurality of embodiments, the C₂₋₆ alkenyl group is a vinyl group, an allyl group, a 1-propenyl group, a 2-propenyl group, a 1-butenyl group, a 2-butenyl group, a 3-butenyl group, a pentenyl group, or a hexenyl group.

The “C₂₋₆ alkynyl group” in the present disclosure means a linear or branched chain alkynyl group having two to six carbon atoms. In one or a plurality of embodiments, the C₂₋₆ alkynyl group is an ethynyl group, a 1-propynyl group, a 2-propynyl group, a butynyl group, a pentynyl group, or a hexynyl group.

The “C₁₋₆ alkoxy group” in the present disclosure means an oxy group to which a C₁₋₆ alkyl group is bonded. In one or a plurality of embodiments, the C₁₋₆ alkoxy group is a methoxy group, an ethoxy group, a 1-propyloxy group, a 2-propyloxy group, a 2-methyl-1-propyloxy group, a 2-methyl-2-propyloxy group, a 1-butyloxy group, a 2-butyloxy group, a 1-pentyloxy group, a 2-pentyloxy group, a 3-pentyloxy group, a 2-methyl-1-butyloxy group, a 3-methyl-1-butyloxy group, a 2-methyl-2-butyloxy group, a 3-methyl-2-butyloxy group, a 2,2-dimethyl-1-propyloxy group, a 1-hexyloxy group, a 2-hexyloxy group, a 3-hexyloxy group, a 2-methyl-1-pentyloxy group, a 3-methyl-1-pentyloxy group, a 4-methyl-1-pentyloxy group, a 2-methyl-2-pentyloxy group, a 3-methyl-2-pentyloxy group, a 4-methyl-2-pentyloxy group, a 2-methyl-3-pentyloxy group, a 3-methyl-3-pentyloxy group, a 2,3-dimethyl-1-butyloxy group, a 3,3-dimethyl-1-butyloxy group, a 2,2-dimethyl-1-butyloxy group, a 2-ethyl-1-butyloxy group, a 3,3-dimethyl-2-butyloxy group, or a 2,3-dimethyl-2-butyloxy group.

The “C₁₋₄ alkoxycarbonyl group” in the present disclosure means a carbonyl group to which a C₁₋₄ alkoxy group is bonded. In one or a plurality of embodiments, the C₁₋₄ alkoxycarbonyl group is a methoxycarbonyl group, an ethoxycarbonyl group, a 1-propyloxycarbonyl group, or a 2 propyloxycarbonyl group.

The “C₁₋₄ alkoxy C₁₋₄ alkyl group” in the present disclosure means a C₁₋₄ alkyl group to which a C₁₋₄ alkoxy group is bonded. In one or a plurality of embodiments, the C₁₋₄ alkoxy C₁₋₄ alkyl group is a methoxyethyl group, or an ethoxymethyl group.

The “cycloalkyl group” in the present disclosure means an alicyclic hydrocarbon group. In one or a plurality of embodiments, the cycloalkyl group is a 3 to 7 membered cycloalkyl group. In one or a plurality of embodiments, the cycloalkyl group may have a single-ring structure, or a double-ring structure. In one or a plurality of embodiments, the cycloalkyl group is a cyclopropyl group, a cyclobutyl group, a cyclopentyl group, or a cyclohexyl group.

The “heterocycloalkyl group” in the present disclosure means a cycloalkyl group in which one or two carbon atoms constituting a ring are substituted with heteroatoms, such as nitrogen atoms, oxygen atoms, or sulfur atoms. In one or a plurality of embodiments, the heterocycloalkyl group is a 3 to 7 membered heterocycloalkyl group. In one or a plurality of embodiments, the heterocycloalkyl group may have a single-ling structure, or a double-ling structure. In one or a plurality of embodiments, the heterocycloalkyl group is an aziridinyl group, an azetidinyl group, a pyrrolidinyl group, a tetrahydrofuranyl group, a tetrahydrothiophenyl group, a piperidinyl group, a piperadizinyl group, a piperazinyl group, a tetrahydropyranyl group, a tetrahydrothiopyranyl group, or a morpholinyl group.

The “aryl group” in the present disclosure means an aromatic hydrocarbon cyclic group. In one or a plurality of embodiments, the aryl group is a 5 to 12 membered aryl group. The aryl group may have a single-ring structure, or a double-ring structure. In one or a plurality of embodiments, the aryl group is a phenyl group, a 1-naphthyl group, or a 2-naphthyl group.

The “heteroaryl group” in the present disclosure means an aryl group in which one or two carbon atoms constituting a ring are substituted with heteroatoms, such as nitrogen atoms, oxygen atoms, or sulfur atoms. In one or a plurality of embodiments, the heteroaryl group is a 5 to 12 membered heteroaryl group. The heteroaryl group may have a single-ring structure, or a double-ring structure. In one or a plurality of embodiments, the heteroaryl group is a furanyl group, a thiophenyl group, a pyrrolyl group, an imidazolyl group, a pyrazolyl group, a triazolyl group, a tetrazolyl group, a thiazolyl group, an oxazolyl group, an isoxazolyl group, an oxadiazolyl group, an oxo-pyridyl group, a thiadiazolyl group, an isothiazolyl group, a pyridyl group, a pyridazyl group, a pyradinyl group, a pyrimidyl group, a quinazolinyl group, a quinolinyl group, an isoquinolinyl group, a benzoimidazoyl group, a benzofuranyl group, a benzothiophenyl group, an indolyl group, or an indazolyl group.

The “nitrogen-containing heteroaryl group” in the present disclosure means a heteroaryl group in which one or two carbon atoms constituting a ring are substituted with nitrogen atoms. In one or a plurality of embodiments, the nitrogen-containing heteroaryl group is a pyridyl group, a pyrrolyl group, an oxazoyl group, an isoxazolyl group, a thiazolyl group, an isothiazolyl group, an indolyl group, an isoindolyl group, a triazoyl group, a pyrazoyl group, a pyridazoyl group, a pyrimidyl group, a pyradinyl group, a quinolinyl group, an isoquinolinyl group, or a benzoimidazoyl group.

The phrase of “substituted or unsubstituted” in the present disclosure means that there is one or a plurality of substituent groups in an arbitrary combination at substitutable sites, or there is no substituent group. In one or a plurality of embodiments, the substituent group is a halogen atom, a cyano group, a trifluoro methyl group, a nitro group, a hydroxyl group, a mercapto group, a formyl group, an oxo group, an imino group, a methylenedioxy group, a C₁₋₆ alkyl group, a C₁₋₆alkoxy group, a benzyloxy group, a C₁₋₆ alkanoyloxy group, an amino group, a mono C₁₋₆ alkylamino group, a di-C₁₋₆ alkylamino group, a carbamoyl group, a C₁₋₆alkylaminocarbonyl group, di-C₁₋₆alkylaminocarbonyl group, a carboxyl group, a C₁₋₆alkoxycarbonyl group, a C₁₋₆alkylthio group, a C₁₋₆ alkylsulfinyl group, a C₁₋₆ alkylsulfonyl group, a C₁₋₆ alkanolyamino group, or a C₁₋₆alkylsulfonamide group. In one or a plurality of embodiments, the halogen atom is fluorine, chlorine, bromine, or iodine.

The “C₁₋₆alkyl group that may have a halogen atom in the substituent group” in the present disclosure means a C₁₋₆ alkyl group in which at least an arbitrary carbon atom is substituted with a halogen atom. In one or a plurality of embodiments, the C₁₋₆alkyl group that may have a halogen atom in the substituent group is a trifluoromethyl group, a difluoromethyl group, or a monofluoromethyl group.

In one or a plurality of embodiments, the compound expressed by Formula (I) is a compound expressed by Formula (II):

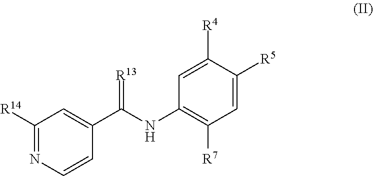

where

R⁴ represents a hydrogen atom; a halogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,

R⁵ represents a hydrogen atom; a halogen atom; an amino group; or an azide group,

R⁷ represents a hydrogen atom; a halogen atom; a diethylamino group; a substituted or unsubstituted nitrogen-containing heterocycloalkyl group; or a substituted or unsubstituted nitrogen-containing heteroaryl group,

R¹³ represents an oxygen atom or a sulfur atom, and

R¹⁴ represents a hydrogen atom; a C₁₋₆ alkyl group; or a C₂₋₆ alkynyl group.

In one or a plurality of embodiments, R⁴ represents a hydrogen atom, a halogen atom, or a C₁₋₆ alkyl group having a substitution with a halogen atom, and preferably represents a hydrogen atom, a fluorine atom, or a trifluoromethyl group.

In one or a plurality of embodiments, R⁵ represents a hydrogen atom, an amino group, or an azide group, and preferably represents a hydrogen atom.

In one or a plurality of embodiments, R⁷ represents a substituted or unsubstituted nitrogen-containing heterocyloalkyl group, or a substituted or unsubstituted nitrogen-containing heteroaryl group; preferably represents a substituted or unsubstituted nitrogen-containing heterocycloalkyl group; and more preferably represents a substituted or unsubstituted piperidinyl group. In one or a plurality of embodiments, examples of R⁷ include the following groups. In the following groups, a binding hand to which wavy lines are attached is a binding part where the group is bonded with the compound expressed by Formula (II):

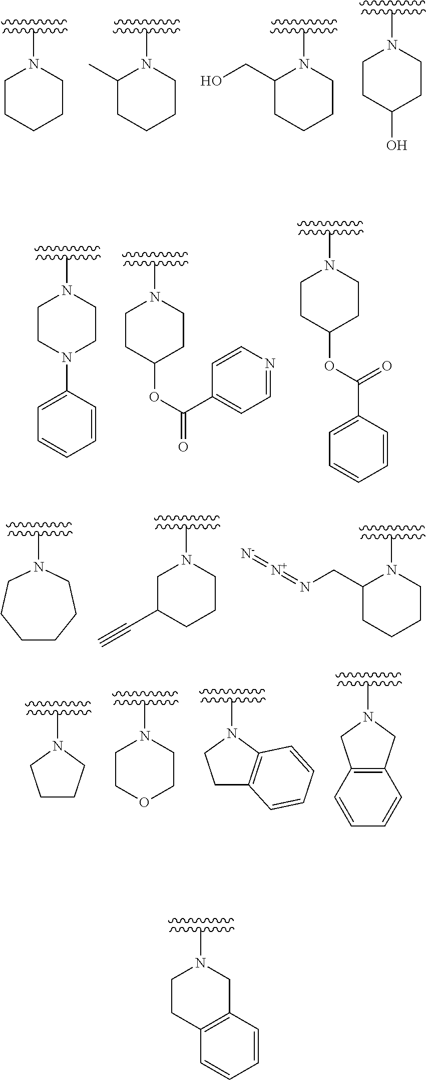

In one or a plurality of embodiments, R¹⁴ represents a hydrogen atom or an ethynyl group, and preferably represents a hydrogen atom.

In one or a plurality of embodiments, examples of the compound expressed by Formula (II) include compounds expressed by the following formulae:

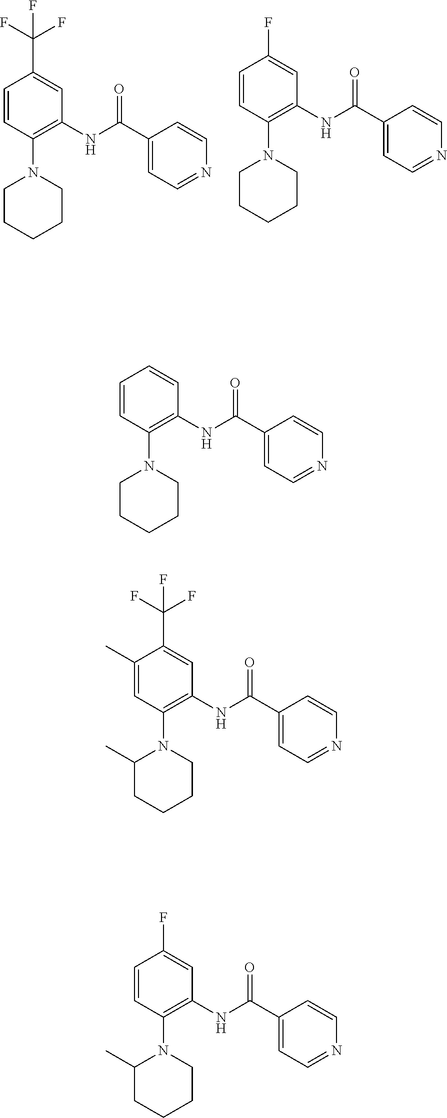

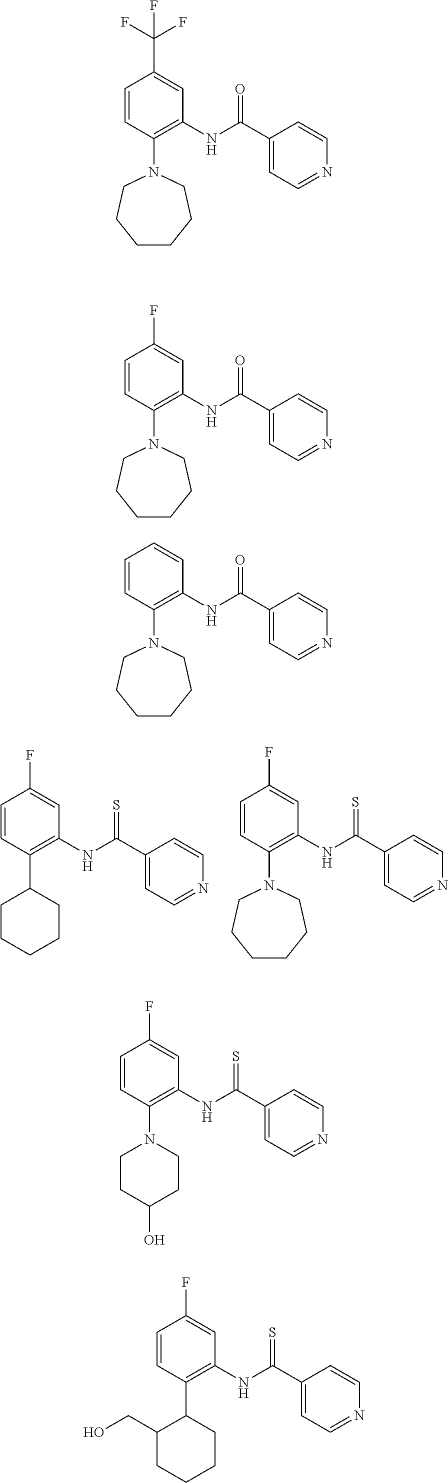

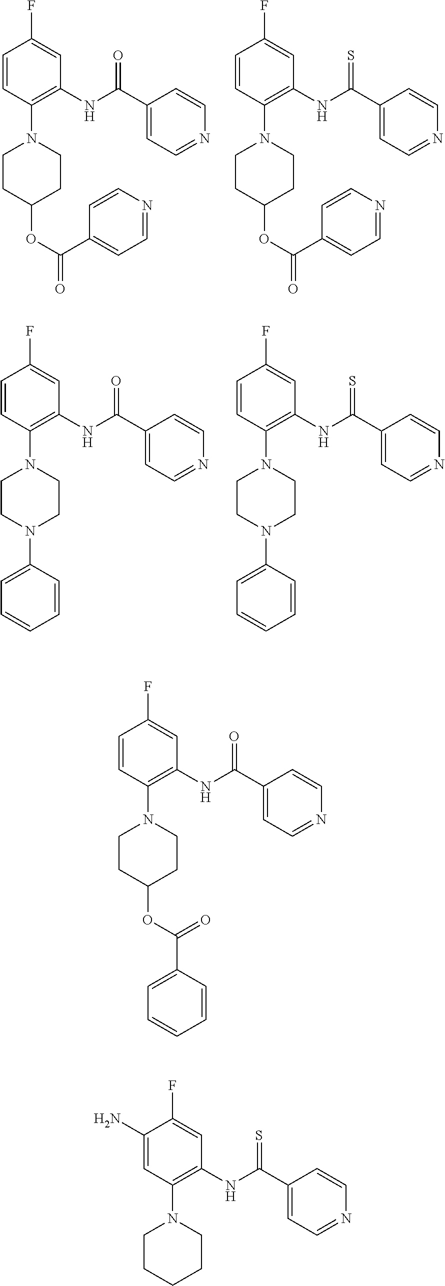

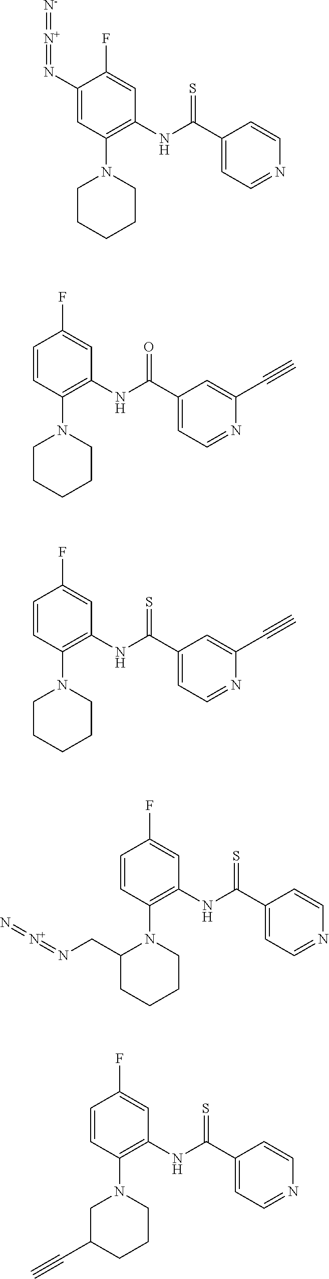

In one or a plurality of embodiments, the above-described compound can have a CDK9 inhibitory activity. Besides, in one or a plurality of embodiments, the above-described compound can exhibit an antiviral effect (antiviral activity). In one or a plurality of embodiments, the above-described compound can exhibit brain permeability. The present disclosure, therefore, in another aspect, relates to a pharmaceutical composition for prevention, treatment, suppression of progression, and/or amelioration of Alzheimer's disease, the pharmaceutical composition containing one, two, or more of the above-described compounds as active ingredients.

In one or a plurality of embodiments, the compound expressed by Formula (I) is a compound expressed by Formula (III):

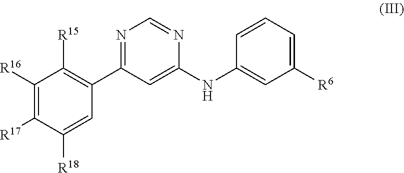

where

R⁶ represents a hydrogen atom; —CSO₂NR¹⁰R¹¹; or —CSO₂R¹²,

where
R¹⁰, R¹¹, and R¹² independently represent a hydrogen atom, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₄ alkoxy C₁₋₄ alkyl group, a substituted or unsubstituted cycloalkyl group, a substituted or unsubstituted heterocycloalkyl group, a substituted or unsubstituted aromatic ring, or a substituted or unsubstituted heteroaryl group,
R¹⁰ and R¹¹ each form a heterocycloalkyl group in combination with a nitrogen atom to which the same is bonded, or
R¹⁰ and R¹¹ each form a heterocycloalkyl group in combination with a nitrogen atom to which the same is bonded, and a sulfur atom to which the nitrogen atom is bonded, and

R¹⁵, R¹⁶, R¹⁷, and R¹⁸ independently represent a hydrogen atom, a halogen atom, a hydroxy group, a C₁₋₄ alkyl group, a C₃₋₇ cycloalkyl group, a C₁₋₄ alkoxy group, a C₁₋₄ alkyl-cycloalkyl group, a C₁₋₄ alkyl-heterocycloalkyl group, an —O-heterocycloalkyl group, a C₂₋₄ alkenyloxy group, —OCF₃, a C₂₋₄ alkanoyl group, a C₁₋₄ alkylsulfonyl group, a mono- and di-(C₁₋₄ alkyl) sulfonamide group, an amino carbonyl group, a mono- and di-(C₁₋₄ alkyl) amino carbonyl group, an aryl-C₁₋₄ alkoxy group, a heteroaryl-C₁₋₄ alkoxy group, a heterocycloalkyl-C₁₋₄ alkoxy group, a heterocycloalkyl-C₁₋₄ alkyl group, a heteroaryl-C₁₋₄ alkyl group, a C₁₋₄ alkyloxy methyl group, a hydroxy-C₁₋₄ alkyloxy methyl group, a cyano group, —COOH, or a C₁₋₄ alkoxycarbonyl group.

In one or a plurality of embodiments, R⁶ represents —CSO₂NR¹⁰R¹¹, where R¹⁰ and R¹¹ independently represent a hydrogen atom, a C₁₋₆ alkyl group, or a C₁₋₄ alkoxy C₁₋₄ alkyl group. In one or a plurality of embodiments, R¹⁰ and R¹¹ independently represent a hydrogen atom, a methyl group, an ethyl group, a 1-propyl group, a 2-propyl group, a tert-butyl group, or a methoxyethyl group. In one or a plurality of embodiments, R¹⁰ represents a methyl group, and R¹¹ represents a hydrogen atom, a methyl group, a 1-propyl group, a 2-propyl group, a tert-butyl group, or a methoxyethyl group.

In one or a plurality of embodiments, R¹⁵, R¹⁶, R¹⁷, and R¹⁸ independently represent a hydrogen atom, a halogen atom, a C₁₋₄ alkoxyl group, or an aryl-C₁₋₄ alkoxy group, and preferably independently represent a hydrogen atom, a fluorine atom, a methoxy group, an ethoxy group, a 1-propyloxy group, a 2-propyloxy group, or a benzyloxy group. In one or a plurality of embodiments, R¹⁶, R¹⁷, and R¹⁸ each represent a hydrogen atom, and R¹⁵ represents a methoxy group or an ethoxy group.

In one or a plurality of embodiments, examples of the compound expressed by Formula (III) include compounds expressed by the following formulae:

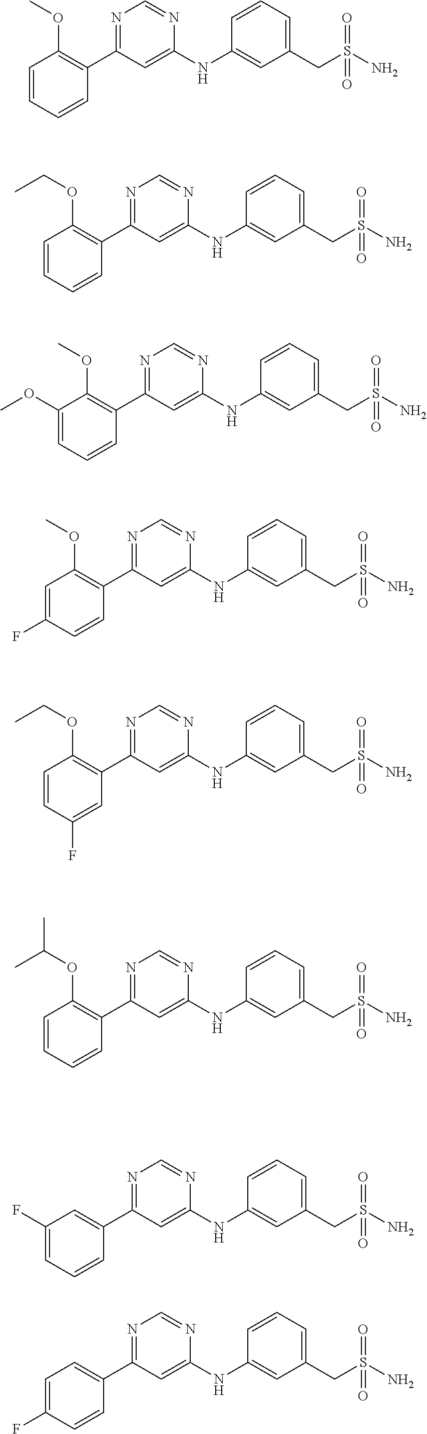

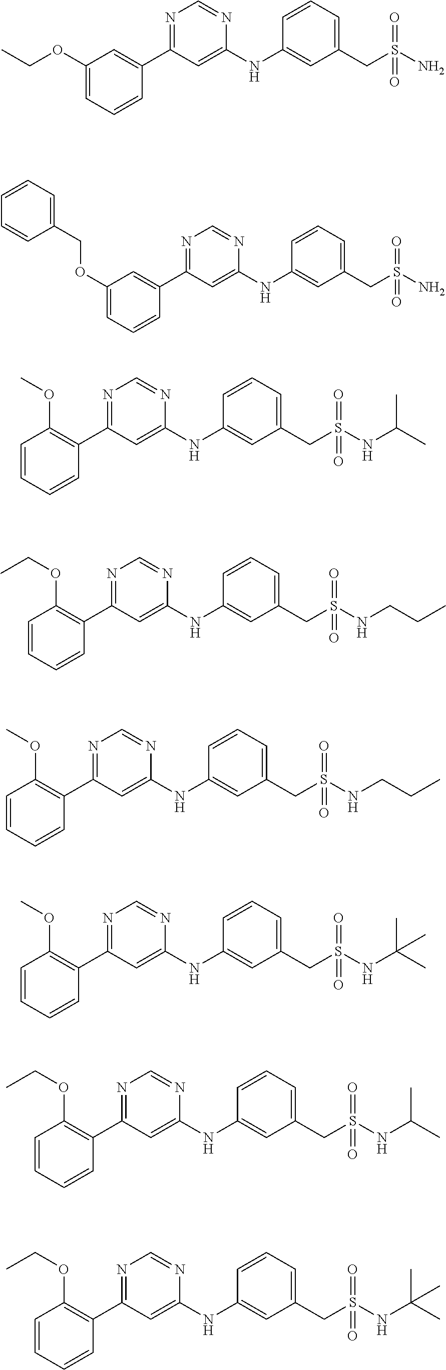

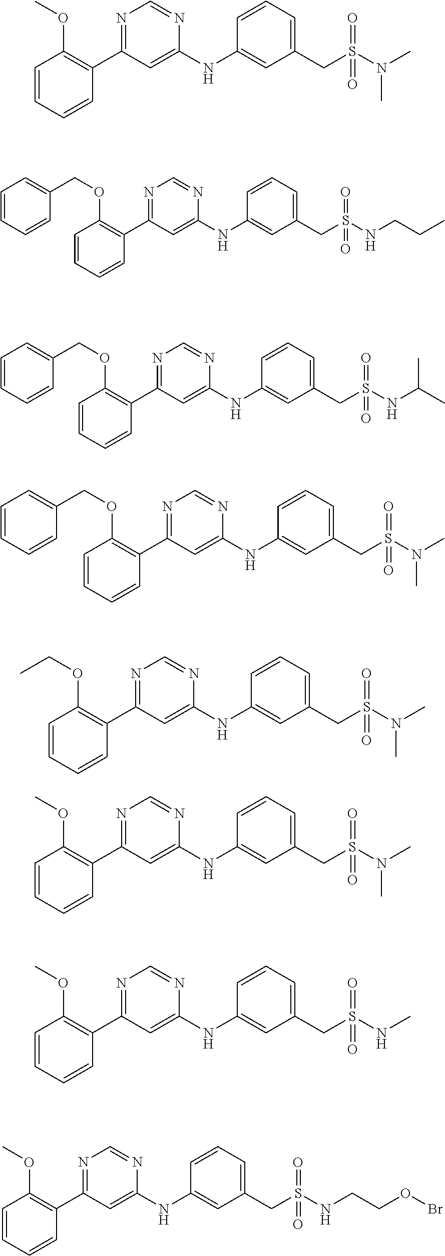

In one or a plurality of embodiments, the above-described compound can have a CDK9 inhibitory activity. Besides, in one or a plurality of embodiments, the above-described compound can exhibit an antiviral activity. The present disclosure, therefore, in another aspect, relates to a pharmaceutical composition for prevention, treatment, suppression of progression, and/or amelioration of Alzheimer's disease, the pharmaceutical composition containing one, two, or more of the above-described compounds as active ingredients.

In one or a plurality of embodiments, the compounds of the present disclosure can be produced with reference to a known producing method or WO2009/020198.

In the present disclosure, the “pharmaceutically acceptable salt” is a pharmacologically and/or pharmaceutically acceptable salt. It is, for example, an inorganic acid salt, an organic acid salt, an inorganic base salt, an organic base salt, an acidic amino acid salt, or a basic amino acid salt. In one or a plurality of embodiments, the inorganic acid salt is a hydrochloride, a hydrobromate, a sulfate, a nitrate, or a phosphate. In one or a plurality of embodiments, the organic acid salt is an acetate, a succinate, a fumarate, a maleate, a tartrate, a citrate, a lactate, a stearate, a benzoate, a methanesulfonate, or a p-toluenesulfonate. In one or a plurality of embodiments, the inorganic base salt is a salt of an alkali metal such as a sodium salt or a potassium salt, an alkali earth metal salt such as a magnesium salt or a calcium salt, an aluminum salt, or an ammonium salt. In one or a plurality of embodiments, the organic base salt is a diethylamine salt, a diethanolamine salt, a meglumine salt, or an N,N′-dibenzylethylenediamine salt. In one or a plurality of embodiments, the acidic amino acid salt is an aspartate, or a glutamate. In one or a plurality of embodiments, the basic amino acid salt is an arginine salt, a lysine salt, or an omithine salt.

In the present disclosure, the “salt of a compound” may encompass a hydrate that can be formed when a compound, left in the atmosphere, absorbs moisture. Further, in the present disclosure, the “salt of a compound” may also encompass a solvate that can be formed when a compound absorbs a solvent of another kind.

In one or a plurality of embodiments, the proportion of the compound contained as an active ingredient in the pharmaceutical composition can be determined appropriately depending on the dosage form, the administration method, and the carrier. In one or a plurality of embodiments, the pharmaceutical composition of the present disclosure can be produced by a conventional method, by adding the compound of the present disclosure at a ratio of 0.01 to 100% (w/w), or 0.1 to 95% (w/w) with respect to the total weight of the formulation.

In one or a plurality of embodiments, the pharmaceutical composition of the present disclosure contains the above-described compound as an active ingredient, and may further contain a pharmaceutically acceptable carrier, preservative, diluent, or excipient, or another pharmaceutically acceptable component.

In one or a plurality of embodiments, the “pharmaceutical composition” in the present disclosure may have a dosage form suitable for an administration form by using the known formulation technology. In one or a plurality of embodiments, regarding the administration form, the pharmaceutical composition can be administered orally in such a dosage form as tablets, capsules, granules, powder, pills, troche, syrups, and liquid formulation. Alternatively, the pharmaceutical composition can be administered parenterally in such a dosage form as injection, liquid formulations, or aerosols. In one or a plurality of embodiments, these formulations can be produced by a known method using additives such as excipients, lubricants, binders, disintegrators, stabilizers, corrigents, and diluents.

In one or a plurality of embodiments, the excipient is a starch such as starch, potato starch, or corn starch; lactose; crystalline cellulose; or calcium hydrogen phosphate. In one or a plurality of embodiments, the lubricant is ethyl cellulose; hydroxypropyl cellulose; hydroxypropyl methylcellulose; shellac; talc; carnauba wax; or paraffin. In one or a plurality of embodiments, the binder is polyvinyl pyrrolidone, macrogol, or any of the compounds similar to those given as examples of the excipient. In one or a plurality of embodiments, the disintegrator is any of compounds similar to those given as examples of the excipient, and chemically modified starches and celluloses such as croscarmellose sodium, sodium carboxymethyl starch, and cross-linked polyvinyl pyrrolidone. In one or a plurality of embodiments, the stabilizer is any of paraoxybenzoic acid esters such as methylparaben and propylparaben; alcohols such as chlorobutanol, benzyl alcohol, and phenylethyl alcohol; benzalkonium chloride; phenols such as phenol and cresol; thimerosal; dehydroacetic acid; and sorbic add. In one or a plurality of embodiments, the corrigent is a commonly used sweetener, acidulant, or flavor.

In one or a plurality of embodiments, in the preparation of a liquid formulation, ethanol, phenol, chlorocresol, purified water, or distilled water can be used as a solvent, and a surface-active agent, a preservative, an isotonizing agent, a pH regulator, an emulsifying agent, or the like can be used as required. In one or a plurality of embodiments, the surface-active agent or the emulsifying agent is polysorbate 80, polyoxyl 40 stearate, or lauromacrogol.

In one or a plurality of embodiments, the pharmaceutical composition of the present disclosure may contain a CDK9 inhibitor other than the compound expressed by Formula (I), (II), or (III).

The method for using the pharmaceutical composition of the present disclosure may differ depending on symptoms, ages, administration methods, etc. In one or a plurality of embodiments, regarding how to use the composition, the pharmaceutical composition can be intermittently or continuously administered orally, percutaneously, submucosally, subcutaneously, intramuscularly, intravascularly, intracerebrally, or intraperitoneally so that the concentration of the above-described compound as an active ingredient in the body is in the range of 100 pM to 1 mM. In a non-limiting embodiment, for oral administration, the pharmaceutical composition may be administered to a subject (e.g., an adult human if the subject is a human) based on the symptom, in a daily dosage of from 0.01 mg/kg body weight to 2000 mg/kg body weight, 0.1 mg/kg body weight to 500 mg/kg body weight, or 0.1 mg/kg body weight to 100 mg/kg body weight, in terms of the compound expressed in Formula (I) described above, at once or in batches. In a non-limiting embodiment, for intravenous administration, the pharmaceutical composition may be administered to a subject (e.g., an adult human if the subject is a human) based on the symptom, in a daily dosage of from 0.001 mg/kg body weight to 50 mg/kg body weight, or 0.01 mg/kg body weight to 50 mg/kg body weight, at once or in batches.

[Method for Preventing, Suppressing the Progression of, Ameliorating, and/or Treating Alzheimer's Disease]

The present disclosure, in one aspect, relates to a method for preventing, treating, suppressing the progression of, and/or ameliorating Alzheimer's disease, the method including administering, to a subject, an effective amount of a compound that is capable of suppressing the activation or growth of virus in the brain as one of factors that cause the onset or progression of Alzheimer's disease. Therefore, the pharmaceutical composition of the present disclosure, and the compound of the present disclosure, in one or a plurality of embodiments, can be used in the prevention, amelioration, and/or treatment of Alzheimer's disease. Therefore, the present disclosure, in another aspect, relates to a method for preventing, treating, suppressing the progression of, and/or ameliorating Alzheimer's disease, the method including administering, to a subject, an effective amount of the pharmaceutical composition of the present disclosure and/or the compound of the present disclosure. The subject is, for example, a human, or an animal other than a human.

In the present disclosure, “the prevention of Alzheimer's disease” encompasses suppressing the onset of Alzheimer's disease, and inhibiting the progression of a pathological condition of mild cognitive impairment caused by Alzheimer's disease. In the present disclosure, “the amelioration of Alzheimer's disease” encompasses stopping the progression of, or minimizing, a pathological condition of mild cognitive impairment caused by Alzheimer's disease. In the present disclosure, “the treatment of Alzheimer's disease” encompasses delaying the progression of, or substantially stopping a pathological condition of mild cognitive impairment caused by Alzheimer's disease.

In one or a plurality of embodiments, the method of the present disclosure may encompass intermittently or continuously, and orally, percutaneously, submucosally, subcutaneously, intramuscularly, intravascularly, intracerebrally, or intraperitoneally administering the above-described compound as an active ingredient so that the concentration of the compound in the body should be at any level in a range of 100 pM to 1 mM. In one or a plurality of embodiments, for oral administration, the method of the present disclosure may encompass administering, to a subject (e.g., an adult human if the subject is a human), the composition in a daily dosage of from 0.01 mg/kg body weight to 2000 mg/kg body weight, 0.1 mg/kg body weight to 500 mg/kg body weight, or 0.1 mg/kg body weight to 100 mg/kg body weight, in terms of the compound expressed in Formula (I) described above, at once or in batches, based on the symptom. In one or a plurality of embodiments, for intravenous administration, the method of the present disclosure may encompass administering, to a subject (e.g., an adult human if the subject is a human), the composition in a daily dosage of from 0.001 mg/kg body weight to 50 mg/kg body weight, or 0.01 mg/kg body weight to 50 mg/kg body weight, at once or in batches, based on the symptom.

The present disclosure, in another aspect, relates to use of the above-described compound for the production of a pharmaceutical composition for prevention, suppression of progression, amelioration, and/or treatment of Alzheimer's disease. The present disclosure, in another aspect, relates to use of the above-described compound for prevention, suppression of progression, amelioration, and/or treatment of Alzheimer's disease.

[Another Aspect]

In one or a plurality of embodiments, the compound of the present disclosure can suppress the accumulation of an Aβ aggregate in the brain. Therefore, in one or a plurality of embodiments, the compound of the present disclosure can be used in the prevention, suppression of progression, amelioration, and/or treatment of Lewy body disease, and Lewy body disease with dementia (Lewy body dementia). Therefore, the present disclosure, in another aspect, relates to a pharmaceutical composition for prevention, suppression of progression, amelioration, and/or treatment of Lewy body disease, and Lewy body disease with dementia (Lewy body dementia), the pharmaceutical composition containing the compound of the present disclosure as an active ingredient, and relates to a method for preventing, suppressing the progression of, ameliorating, and/or treating Lewy body disease, and Lewy body disease with dementia (Lewy body dementia), the method including administering an effective amount of the compound of the present disclosure to a subject.

In one or a plurality of embodiments, the compound of the present disclosure has a CDK9 inhibitory activity and intracerebral transferability. Therefore, in one or a plurality of embodiments, the compound of the present disclosure can be used in the prevention, suppression of progression, amelioration, and/or treatment of viral infection in the brain and/or a neurodegenerative disease caused by the activation of virus in the brain. Therefore, the present disclosure, in another aspect, relates to a pharmaceutical composition for prevention, suppression of progression, amelioration, and/or treatment of viral infection in the brain and/or a neurodegenerative disease caused by the activation of virus in the brain, the pharmaceutical composition containing the compound of the present disclosure as an active ingredient, and relates to a method for preventing, suppressing the progression of, ameliorating, and/or treating viral infection in the brain and/or a neurodegenerative disease caused by the activation of virus in the brain, the method including administering an effective amount of the compound of the present disclosure to a subject.

The present disclosure, in another aspect, relates to a composition and a pharmaceutical composition for the inhibition of CDK9 activity in the brain, the composition and the pharmaceutical composition containing the compound of the present disclosure as an active ingredient, and relates to a method for inhibiting CDK9 activity in the brain, the method including administering an effective amount of the compound of the present disclosure to a subject.

The present disclosure, in another aspect, relates to a composition and a pharmaceutical composition for the suppression of the activation and/or growth of virus in the brain, the composition and the pharmaceutical composition containing the compound of the present disclosure as an active ingredient, and relates to a method for suppressing the activation and/or growth of virus in the brain, the method including administering an effective amount of the compound of the present disclosure to a subject.

In the present disclosure, the subject is, for example, an animal such as a human or a mammal other than a human.

In other words, the present disclosure may relate to one or a plurality of embodiments described below:

[1] A pharmaceutical composition for prevention, treatment, suppression of progression, and/or amelioration of Alzheimer's disease,
the pharmaceutical composition containing, as an active ingredient, a compound capable of suppressing activation or growth of virus in the brain as one of factors that cause onset or progression of Alzheimer's disease.
[2] The pharmaceutical composition according to [1],

wherein the compound is a compound capable of suppressing growth of DNA virus and/or retrovirus by inhibiting CDK9.

[3] The pharmaceutical composition according to [2],

wherein the compound can pass through blood-brain barrier.

[4] The pharmaceutical composition according to any one of [1] to [3],

wherein the compound is a compound expressed by Formula (I) below, or a pharmaceutically acceptable salt of the compound:

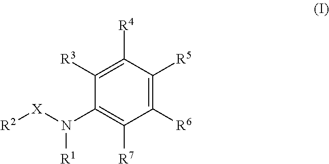

where

R¹ represents a hydrogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,

X represents —C(═O)—; —C(═S)—; —SO₂—; —C(═S)NHC(═O)—; —C(═O)NHC(═S)—; or

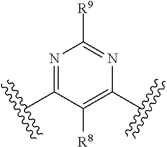

where R⁸ and R⁹ independently represents a hydrogen atom; a substituted or unsubstituted C₁₋₆ alkyl group; a C₂₋₆ alkenyl group; a C₂₋₆ alkynyl group; a halogen atom; —CN; —NH₂; or —NO₂,

R² represents a substituted or unsubstituted C₁₋₆ alkyl group; a substituted or unsubstituted C₂₋₆ alkenyl group; a substituted or unsubstituted C₂₋₆ alkynyl group; a substituted or unsubstituted aryl group; or a substituted or unsubstituted nitrogen-containing heteroaryl group,

R³ represents a hydrogen atom; a substituted or unsubstituted C₁₋₆ alkyl group; a C₂₋₆ alkenyl group; a C₂₋₆ alkynyl group; a halogen atom; —CN; —NH₂; or —NO₂,

R⁴ represents a hydrogen atom; a halogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,

R⁵ represents a hydrogen atom; a halogen atom; an amino group; or an azide group,

R⁶ represents a hydrogen atom; —CSO₂NR¹⁰R¹¹; or —CSO₂R¹²,
where
   R¹⁰, R¹¹, and R¹² independently represent a hydrogen atom, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₄ alkoxy C₁₋₄ alkyl group, a substituted or unsubstituted cycloalkyl group, a substituted or unsubstituted heterocycloalkyl group, a substituted or unsubstituted aromatic ring, or a substituted or unsubstituted heteroaryl group,
   R¹⁰ and R¹¹ each form a heterocycloalkyl group in combination with a nitrogen atom to which the same is bonded, or
   R¹⁰ and R¹¹ each form a heterocycloalkyl group in combination with a nitrogen atom to which the same is bonded, and a sulfur atom to which the nitrogen atom is bonded, and

R⁷ represents a hydrogen atom; a halogen atom; a diethylamino group; a substituted or unsubstituted nitrogen-containing heterocycloalkyl group; or a substituted or unsubstituted nitrogen-containing heteroaryl group.

[5] A pharmaceutical composition for prevention, treatment, suppression of progression, and/or amelioration of Alzheimer's disease, the pharmaceutical composition containing, as an active ingredient, a compound expressed by Formula (II) below or a pharmaceutically acceptable salt thereof, and/or a compound expressed by Formula (III) below or a pharmaceutically acceptable salt thereof:

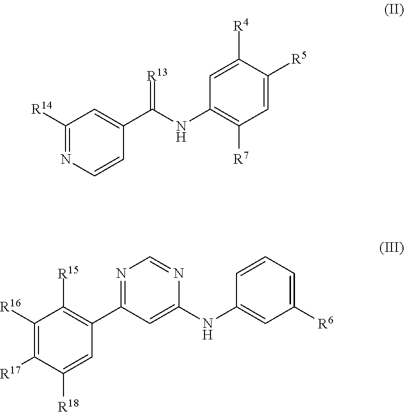

where,

### in Formula (II),

R⁴ represents a hydrogen atom; a halogen atom; a substituted or unsubstituted alkyl group; a substituted or unsubstituted C₂₋₆ alkenyl group; a substituted or unsubstituted C₂₋₆ alkynyl group; or a substituted or unsubstituted aryl group,

R⁵ represents a hydrogen atom; a halogen atom; an amino group; or an azide group,

R⁷ represents a hydrogen atom; a halogen atom; a diethylamino group; a substituted or unsubstituted nitrogen-containing heterocycloalkyl group; or a substituted or unsubstituted nitrogen-containing heteroaryl group,

R¹³ represents an oxygen atom or a sulfur atom, and

R¹⁴ represents a hydrogen atom; a C₁₋₆ alkyl group; or a C₂₋₆ alkynyl group, and in Formula (III),

R⁶ represents a hydrogen atom; —CSO₂NR¹⁰R¹¹; or —CSO₂R¹²,
where
   R¹⁰, R¹¹, and R¹² independently represent a hydrogen atom, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₄ alkoxy C₁₋₄ alkyl group, a substituted or unsubstituted cycloalkyl group, a substituted or unsubstituted heterocycloalkyl group, a substituted or unsubstituted aromatic ring, or a substituted or unsubstituted heteroaryl group,
   R¹⁰ and R¹¹ each form a heterocycloalkyl group in combination with a nitrogen atom to which the same is bonded, or
   R¹⁰ and R¹¹ each form a heterocycloalkyl group in combination with a nitrogen atom to which the same is bonded, and a sulfur atom to which the nitrogen atom is bonded, and

R¹⁵, R¹⁶, R¹⁷, and R¹⁶ independently represent a hydrogen atom, a halogen atom, a hydroxy group, a C₁₋₄ alkyl group, a C₃₋₇ cycloalkyl group, a C₁₋₄ alkoxy group, a C₁₋₄ alkyl-cycloalkyl group, a C₁₋₄ alkyl-heterocycloalkyl group, an —O-heterocycloalkyl group, a C₂₋₄ alkenyloxy group, —OCF₃, a C₂₋₄ alkanoyl group, a C₁₋₄ alkylsulfonyl group, a mono- and di-(C₁₋₄ alkyl) sulfonamide group, an amino carbonyl group, a mono- and di-(C₁₋₄ alkyl) amino carbonyl group, an aryl-C₁₋₄ alkoxy group, a heteroaryl-C₁₋₄ alkoxy group, a heterocycloalkyl-C₁₋₄ alkoxy group, a heterocycloalkyl-C₁₋₄ alkyl group, a heteroaryl-C₁₋₄ alkyl group, a C₁₋₄ alkyloxy methyl group, a hydroxy-C₁₋₄ alkyloxy methyl group, a cyano group, —COOH, or a C₁₋₄ alkoxycarbonyl group.

[6] A method for preventing, treating, suppressing the progression of, and/or ameliorating Alzheimer's disease,

the method including administering, to a subject, an effective amount of a compound capable of suppressing the activation or growth of virus in the brain as one of factors that cause the onset or progression of Alzheimer's disease.

[7] The method according to [6],

wherein the compound is the compound according to [4] or [5].

### Example

Hereinafter, although the following description describes the present disclosure in more detail by way of examples, these are illustrative, and the present disclosure is not limited to these examples. Note that all of the documents cited in the present disclosure are incorporated as a part of the present disclosure.

[Assay of CDK9 Inhibitory Activity]

Tests for confirming CDK9 inhibitory activity were carried out on Compounds 1 to 5 shown below.

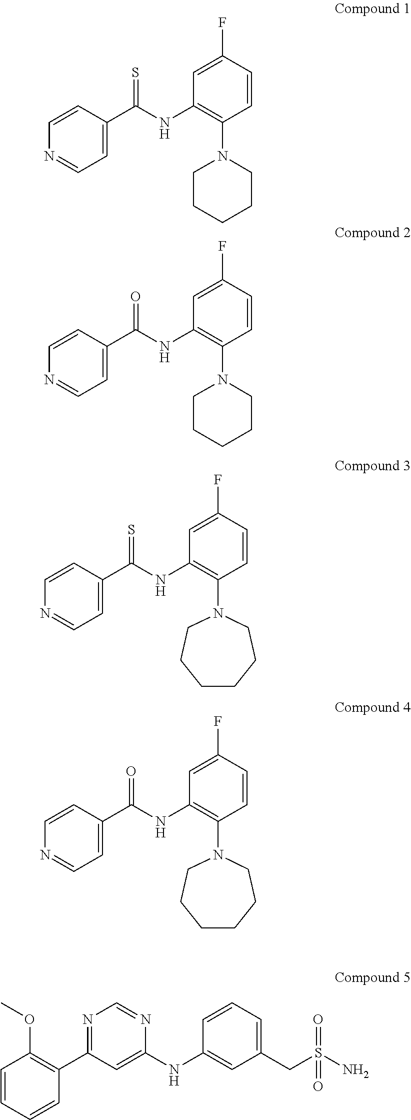

The tests for confirming CDK9 inhibitory activity were carried out by the following procedure according to Off-chip Mobility Shift Assay (MSA).

1) Test substance solutions (4×) each containing one of Compounds 1 to 5 were prepared with use of an assay buffer (20 mM HEPES, 0.01% Triton X-100, 1 mM DTT, pH7.5). A substrate/ATP/metal solution (4×) was prepared with use of a kit buffer (20 mM HEPES, 0.01% Triton X-100, 5 mM DTT, pH7.5). A kinase solution (2×) was prepared with use of an assay buffer.

2) The test substance solutions, the substrate/ATP/metal solution, and the kinase solution were mixed in wells of a polypropylene plate, and were caused to react at room temperature for one hour.

3) A termination buffer (QuickScout Screening Assist MSA; Cama Biosciences Inc.)

was added thereto to stop the reaction.

4) Substrate peptides and phosphorylated peptides in the reaction solutions were separated and quantified in LabChip™ System (PerkinElmer Co., Ltd.).

5) Kinase reaction activities were evaluated based on a product ratio (P/(P+S)) calculated from a peak height of the substrate peptide (S) and a peak height of the phosphorylated peptide (P).

IC50 values with respect to CDK9/CycT1 were calculated from what is described above, as follows: Compound 1 had an IC50 value of 5.8 μM; Compound 2, 13.1 μM; Compound 3, 13.3 μM; Compound 4, 13.7 μM; and Compound 5, 84 nM. Therefore, Compounds 1 to 5 had CDK9 inhibitory activity.

[Testing the Penetration of the Compound Through the Blood-Brain Barrier]

Brain permeability (intracerebral transferability) of Compound 1 was evaluated.

¹⁴C-labeled Compound 1 was intravenously administered once in a dose of 5 mg/kg (3.52 MBq/kg) to rats, and histological distribution (biodistribution) thereof was studied. The results are shown in Table 1.

Table 1 shows exemplary distribution of radioactivity in tissues as a result of single intravenous administration of ¹⁴C-labeled Compound 1. As shown in Table 1, all of radioactive concentrations in the cerebellum, the cerebrum, and the pituitary gland at 5 minutes after the administration were higher than the radioactive concentration in plasma. Therefore, it was confirmed that Compound 1 is capable of penetrating the brain.

TABLE 1
Radioactivity concentration (ng eq. of Compound 1/g or mL)

| Tissue | 5 min | 6 h | 24 h | 72 h | 168 h |
|---|---|---|---|---|---|
| Plasma | 2644.34 | 1132.96 | 121.88 | 8.73 | 3.46 |
| Blood | 2064.57 | 959.54 | 109.67 | 12.94 | ND |
| Cerebrum | 12122.17 | 853.47 | 89.01 | 7.26 | ND |
| Cerebellum | 12499.69 | 905.96 | 102.6 | 12.62 | ND |
| Pituitary gland | 10215.59 | 849.11 | 66.5 | ND | ND |
| Eyeball | 1785.93 | 960.39 | 117.64 | 7.03 | ND |
| Harderian gland | 12633.83 | 1418.19 | 131.47 | 8.95 | ND |
| Thyroid gland | 9550.71 | 2369.71 | 793.99 | 298.2 | 125.76 |
| Mandibular gland | 13355.34 | 995.48 | 115.51 | 8.87 | ND |
| Mandibular lymph node | 5693.66 | 1018.12 | 108.82 | 8.13 | ND |
| Thymus | 5034.15 | 954.13 | 97.42 | 5.22 | ND |
| Heart | 8604.53 | 912.54 | 105.78 | 10.01 | ND |
| Lung | 7645.96 | 1247.84 | 175.43 | 33.55 | 12.98 |
| Liver | 29518.18 | 3033.96 | 740.19 | 180.72 | 48.2 |
| Kidney | 13230.64 | 2138.26 | 264.55 | 31.5 | 12.02 |
| Adrenal gland | 47334.91 | 1517.3 | 195.16 | 23.21 | ND |
| Spleen | 6622.6 | 944.65 | 113.01 | 9.68 | ND |
| Pancreas | 14356.66 | 1206.84 | 105.62 | 7.37 | ND |
| Fat | 1181.88 | 5031.8 | 1665.35 | 112.35 | ND |
| Brown fat | 8467.9 | 6412.21 | 217.43 | 11.3 | ND |
| Skeletal muscle | 2479.87 | 838.23 | 97.94 | 10.11 | ND |
| Skin | 3889.26 | 2084.85 | 150.1 | 23.26 | 6.57 |
| Bonemarrow | 6109.89 | 900.75 | 106.7 | ND | ND |
| Aorta | 1995.27 | 730.03 | 76.22 | ND | ND |
| Testis | 2762.22 | 1217.12 | 128.05 | 8.72 | ND |
| Epididymis | 2568.88 | 1809.29 | 312.66 | 21.99 | ND |
| Prostate gland | 5072.42 | 1268.5 | 114.45 | 7.62 | ND |
| Stomach | 8261.97 | 2309.97 | 227.12 | ND | ND |
| Small intestine | 6555.86 | 1979.83 | 156.71 | 9.85 | ND |
| Cecum | 5555.55 | 3979.48 | 559.36 | 17.15 | ND |
| Large intestine | 5132.52 | 2429.43 | 388.61 | 9.66 | ND |
| Urinary bladder | 2474.13 | 7563.63 | 356.22 | 19.22 | ND |

ND: not detected, n = 3

[Antiviral Test Against HHV-6A]

An antiviral test against HHV-6A using Compound 1 was performed by the following procedure.

Virus strain: Human Herpesvirus Type 6A (HHV-6A)

Cell line: Sup-T1 cells

Medium: RPMI-1640+10% FBS+1% penicillin/streptomycin

Detection method: TaqMan PCR

### [Experiment Procedure]

Sup-T1 cells were cultured at a concentration of 1×10⁶/ml, Compound 1, 3 μM, was added, and one hour after, HHV-6A virus (1×10⁴ infectious units) were sensitized (or unsensitized cells were prepared as a negative control).

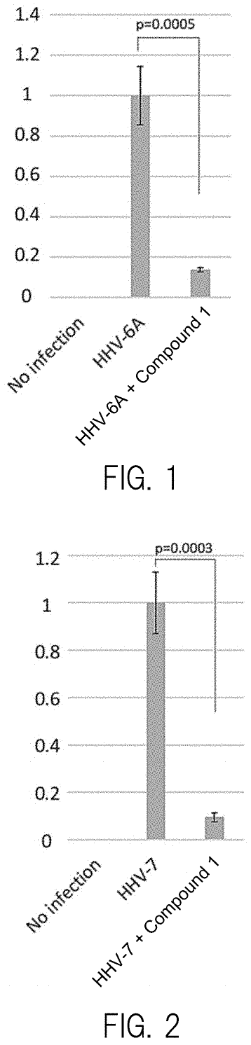

After culturing at 37° C. for two hours, the medium was replaced, and Compound 1 was added again. Three days after, 500 μl of the medium was sampled, and HHV-6A virus was quantified by TaqMan PCR. Statistical analysis was performed by Student's t-test. The results are shown in the table below and FIG. 1.

### [Results]

As a result of the quantitative analysis, an effect of suppressing growth of HHV-6A in the Compound 1-treated group was confirmed, whereby the antiviral effect of Compound 1 against HHV-6A was confirmed (the table shows measured values, and the graph of FIG. 1 shows a comparison of fold-change against amounts of HHV-6A virus).

TABLE 2
Results of antiviral test against HHV-6A

| Samples | HHV-6A copy number | SD |
|---|---|---|
| No infection | 0 | 0 |
| HHV-6A | 49961.56 | 7179.13 |
| HHV-6A + Compound 1 | 6767.59 | 454.61 |

[Antiviral Test Against HHV-7]

An antiviral test against HHV-7 using Compound 1 was performed by the following procedure.

Virus strain: Human Herpesvirus Type 7 (HHV-7)

Cell line: Sup-T1 cells

Medium: RPMI-1640+10% FBS+1% penicillin/streptomycin

Detection method: TaqMan PCR

### [Experiment Procedure]

Sup-T1 cells were cultured at a concentration of 1×10⁶/ml, Compound 1, 10 μM, was added, and one hour after, HHV-7 virus (1×10⁴ infectious units) were sensitized (or unsensitized cells were prepared as a negative control).

After culturing at 37° C. for two hours, the medium was replaced, and Compound 1 was added again. Five days after, 500 μl of the medium was sampled, and HHV-7 virus was quantified by TaqMan PCR. Statistical analysis was performed by Student's t-test. The results are shown in the table below and FIG. 2.

### [Results]

As a result of the quantitative analysis, an effect of suppressing growth of HHV-7 was confirmed in the Compound 1-treated group, whereby the antiviral effect of Compound 1 against HHV-7 was confirmed (the table shows measured values, and the graph of FIG. 2 shows a comparison of fold-change against amounts of HHV-7 virus).

TABLE 3
Results of antiviral test against HHV-7

| Samples | HHV-7 copy number | SD |
|---|---|---|
| No infection | 0 | 0 |
| HHV-7 | 19285.28 | 2493.06 |
| HHV-7 + Compound 1 | 1844.89 | 361.31 |

1. A pharmaceutical composition for prevention, treatment, suppression of progression, and/or amelioration of Alzheimer's disease,
the pharmaceutical composition containing, as an active ingredient, a compound capable of suppressing activation or growth of virus in brain as one of factors that cause onset or progression of Alzheimer's disease.

2. The pharmaceutical composition according to claim 1,
wherein the compound is a compound capable of suppressing growth of DNA virus and/or retrovirus by inhibiting CDK9.

3. The pharmaceutical composition according to claim 2,
wherein the compound can pass through blood-brain barrier.

4. The pharmaceutical composition according to claim 1,
wherein the compound is a compound expressed by Formula (I) below, or a pharmaceutically acceptable salt of the compound: 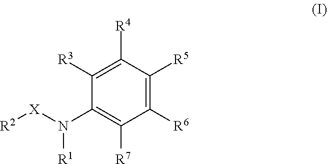
where
R¹ represents a hydrogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,
X represents —C(═O)—; —C(═S)—; —SO₂—; —C(═S)NHC(═O)—; —C(═O)NHC(═S)—; or 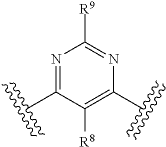
where R⁸ and R⁹ independently represents a hydrogen atom; a substituted or unsubstituted C₁₋₆ alkyl group; a C₂₋₆ alkenyl group; a C₂₋₆ alkynyl group; a halogen atom; —CN; —NH₂; or —NO₂,
R² represents a substituted or unsubstituted C₁₋₆ alkyl group; a substituted or unsubstituted C₂₋₆ alkenyl group; a substituted or unsubstituted C₂₋₆ alkynyl group; a substituted or unsubstituted aryl group; or a substituted or unsubstituted nitrogen-containing heteroaryl group,
R³ represents a hydrogen atom; a substituted or unsubstituted C₁₋₆ alkyl group; a C₂₋₆ alkenyl group; a C₂₋₆ alkynyl group; a halogen atom; —CN; —NH₂; or —NO₂,
R⁴ represents a hydrogen atom; a halogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,
R⁵ represents a hydrogen atom; a halogen atom; an amino group; or an azide group,
R⁶ represents a hydrogen atom; —CSO₂NR¹⁰R¹¹; or —CSO₂R¹²,
where
R¹⁰, R¹¹, and R¹² independently represent a hydrogen atom, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₄ alkoxy C₁₋₄ alkyl group, a substituted or unsubstituted cycloalkyl group, a substituted or unsubstituted heterocycloalkyl group, a substituted or unsubstituted aromatic ring, or a substituted or unsubstituted heteroaryl group,
R¹⁰ and R¹¹ each form a heterocycloalkyl group in combination with a nitrogen atom to which the same is bonded, or
R¹⁰ and R¹¹ each form a heterocycloalkyl group in combination with a nitrogen atom to which the same is bonded, and a sulfur atom to which the nitrogen atom is bonded, and
R⁷ represents a hydrogen atom; a halogen atom; a diethylamino group; a substituted or unsubstituted nitrogen-containing heterocycloalkyl group; or a substituted or unsubstituted nitrogen-containing heteroaryl group.

5. A pharmaceutical composition for prevention, treatment, suppression of progression, and/or amelioration of Alzheimer's disease,
the pharmaceutical composition containing, as an active ingredient, a compound expressed by Formula (II) below or a pharmaceutically acceptable salt thereof, and/or a compound expressed by Formula (III) below or a pharmaceutically acceptable salt thereof: 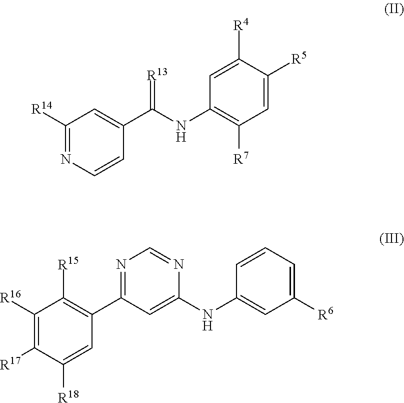
where,
in Formula (II),
R⁴ represents a hydrogen atom; a halogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,
R⁵ represents a hydrogen atom; a halogen atom; an amino group; or an azide group,
R⁷ represents a hydrogen atom; a halogen atom; a diethylamino group; a substituted or unsubstituted nitrogen-containing heterocycloalkyl group; or a substituted or unsubstituted nitrogen-containing heteroaryl group,
R¹³ represents an oxygen atom or a sulfur atom, and
R¹⁴ represents a hydrogen atom; a C₁₋₆ alkyl group; or a C₂₋₆ alkynyl group, and
in Formula (III),
R⁶ represents a hydrogen atom; —CSO₂NR¹⁰R¹¹; or —CSO₂R¹²,
where
R¹⁰, R¹¹, and R¹² independently represent a hydrogen atom, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₄ alkoxy C₁₋₄ alkyl group, a substituted or unsubstituted cycloalkyl group, a substituted or unsubstituted heterocycloalkyl group, a substituted or unsubstituted aromatic ring, or a substituted or unsubstituted heteroaryl group,
R¹⁰ and R¹¹ each form a heterocycloalkyl group in combination with a nitrogen atom to which the same is bonded, or
R¹⁰ and R¹¹ each form a heterocycloalkyl group in combination with a nitrogen atom to which the same is bonded, and a sulfur atom to which the nitrogen atom is bonded, and
R¹⁵, R¹⁶, R¹⁷, and R¹⁸ independently represent a hydrogen atom, a halogen atom, a hydroxy group, a C₁₋₄ alkyl group, a C₃₋₇ cycloalkyl group, a C₁₋₄ alkoxy group, a C₁₋₄ alkyl-cycloalkyl group, a C₁₋₄ alkyl-heterocycloalkyl group, an —O-heterocycloalkyl group, a C₂₋₄ alkenyloxy group, —OCF₃, a C₂₋₄ alkanoyl group, a C₁₋₄ alkylsulfonyl group, a mono- and di-(C₁₋₄ alkyl) sulfonamide group, an amino carbonyl group, a mono- and di-(C₁₋₄ alkyl)amino carbonyl group, an aryl-C₁₋₄ alkoxy group, a heteroaryl-C₁₋₄ alkoxy group, a heterocycloalkyl-C₁₋₄ alkoxy group, a heterocycloalkyl-C₁₋₄ alkyl group, a heteroaryl-C₁₋₄ alkyl group, a C₁₋₄alkyloxy methyl group, a hydroxy-C₁₋₄ alkyloxy methyl group, a cyano group, —COOH, or a C₁₋₄ alkoxycarbonyl group.

6. A method for preventing, treating, suppressing the progression of, and/or ameliorating Alzheimer's disease,
the method including administering, to a subject, an effective amount of a compound capable of suppressing activation or growth of virus in brain as one of factors that cause onset or progression of Alzheimer's disease.

7. The method according to claim 6,
wherein the compound is a compound expressed by Formula (I) below, or a pharmaceutically acceptable salt of the compound: 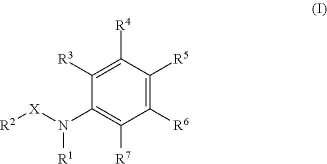
where
R¹ represents a hydrogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,
X represents —C(═O)—; —C(═S)—; —SO₂—; —C(═S)NHC(═O)—; —C(═O)NHC(═S)—; or 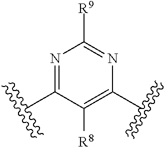
where R⁸ and R⁹ independently represents a hydrogen atom; a substituted or unsubstituted C₁₋₆ alkyl group; a C₂₋₆ alkenyl group; a C₂₋₆ alkynyl group; a halogen atom; —CN; —NH₂; or —NO₂,
R² represents a substituted or unsubstituted C₁₋₆ alkyl group; a substituted or unsubstituted C₂₋₆ alkenyl group; a substituted or unsubstituted C₂₋₆ alkynyl group; a substituted or unsubstituted aryl group; or a substituted or unsubstituted nitrogen-containing heteroaryl group,
R³ represents a hydrogen atom; a substituted or unsubstituted C₁₋₆ alkyl group; a C₂₋₆ alkenyl group; a C₂₋₆ alkynyl group; a halogen atom; —CN; —NH₂; or —NO₂,
R⁴ represents a hydrogen atom; a halogen atom; a substituted or unsubstituted C₁₋₆ alkyl group; a substituted or unsubstituted C₂₋₆ alkenyl group; a substituted or unsubstituted C₂₋₆ alkynyl group; or a substituted or unsubstituted aryl group,
R⁵ represents a hydrogen atom; a halogen atom; an amino group; or an azide group,
R⁶ represents a hydrogen atom; —CSO₂NR¹⁰R¹¹; or —CSO₂R¹²,
where
R¹⁰, R¹¹, and R¹² independently represent a hydrogen atom, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₄ alkoxy C₁₋₄ alkyl group, a substituted or unsubstituted cycloalkyl group, a substituted or unsubstituted heterocycloalkyl group, a substituted or unsubstituted aromatic ring, or a substituted or unsubstituted heteroaryl group,
R¹⁰ and R¹¹ each form a heterocycloalkyl group in combination with a nitrogen atom to which the same is bonded, or
R¹⁰ and R¹¹ each form a heterocycloalkyl group in combination with a nitrogen atom to which the same is bonded, and a sulfur atom to which the nitrogen atom is bonded, and
R⁷ represents a hydrogen atom; a halogen atom; a diethylamino group; a substituted or unsubstituted nitrogen-containing heterocycloalkyl group; or a substituted or unsubstituted nitrogen-containing heteroaryl group.
